(19) United States
(12) Patent Application Publication
(10) Pub. No.: US 2016/0250487 A1
(43) Pub. Date: Sep. 1, 2016

(54) Device and Method for Controlling an Implanted Medical Device

(71) Applicants: Joseph A. Hare, Wenatchee, WA (US); Gregory Eberhart, Wenatchee, WA (US); Robert Parton, Arcata, CA (US); Christopher W. Kocher, Wenatchee, WA (US)

(72) Inventors: Joseph A. Hare, Wenatchee, WA (US); Gregory Eberhart, Wenatchee, WA (US); Robert Parton, Arcata, CA (US); Christopher W. Kocher, Wenatchee, WA (US)

(21) Appl. No.: 14/632,206

(22) Filed: Feb. 26, 2015

(51) Int. Cl. A61N 1/372; A61N 1/362; A61N 1/39

(52) U.S. Cl. CPC A61N 1/37223; A61N 1/3993; A61N 1/362

(57) ABSTRACT

A device for controlling an implanted medical device [IMD] is disclosed and which includes a housing; a magnet mounted within the housing and which generates a source of electromagnetic radiation which is effective in changing the operational condition of the IMD; an electrical sensor borne by the housing and which detects a source of electromagnetic radiation emitted by the IMD, locates the IMD within a patient's body, and identifies the type of IMD within the patient's body; a signaling assembly borne by the housing to give a signal to a user; and a source of electrical power mounted on the housing and which selectively electrically energizes the device.

TECHNICAL FIELD

The present invention relates to a device and an accompanying method for allowing a medical professional to change the operational status of an implanted medical device such that the implanted medical device is not affected during a subsequent surgery.

### BACKGROUND OF THE INVENTION

Cautery devices are commonly used during a wide variety of medical surgeries and procedures. Often cautery devices are used on patients who already have an implanted, electrically energized medical device (IMD) such as a cardiac pacemaker or a cardiac defibrillator, or are used during the implantation of an IMD. As further described in detail in Lure et. al. reference (U.S. Pub. No. 2011/0160782) there is often concern that the use of cautery devices, especially unipolar cautery devices, has the potential to interfere with normal functional operation of a patient's IMD's. Specifically, there are legitimate concerns that the IMD's programming, function, and circuitry may be dangerously influenced by the electrical signals generated by the cautery device.

It is recommended by many IMD manufacturers and medical specialists that an IMD be placed into a so-called “safety mode” prior to surgery to preclude the IMD from detecting the external electrical signal of a cautery device. Traditionally IMD's were placed into safety mode by use of a magnetic field communication device, as all well-known IMD's are manufactured to switch to a safety mode in the presence of a magnetic field. With varying degrees of success this change of the operational status or condition of the IMD has been accomplished prior to surgery by the use of pre-surgical reprogramming devices, which are approximately the size of a laptop computer, and which are further connected to a programming member which is much like a computer mouse. More recently similar computer devices have used wireless radio frequencies (RF) to communicate with IMD's that are manufactured to have the ability to receive RF communications.

Such pre-surgical reprogramming of the respective IMD's includes disabling therapeutic, or sensing functions of the IMD, and leaving the patient without the defibrillation or pacing benefits of the IMD until the device is reprogrammed following the completion of the surgery. As should be understood, programming and re-programming requires considerable human resources, and may be inconvenient in emergency situations as well as during invasive procedures in which the manufacturer representatives or specialized implantable device professionals are not immediately available. Further downside risks associated with the current practice includes the potential for miscommunication between medical personnel as to the status of the patient's IMD because reprogramming typically occurs outside of the operating theatre, the potential for an unanticipated and non-recognizable change in the status of a patient's IMD during surgery, the size and costs associated with manufacturing and operating the programming device, and the risks associated with leaving a patient's IMD in a safety mode following the surgical intervention, and thus placing the patient at an increased health risk should an adverse coronary event unexpectedly take place.

When IMD programming representatives are unavailable, such as during emergency situations, or at remotely located health care facilities, there are times when medical professionals attempt to place a patient's IMD in to a safety mode by creating a magnetic field which is located in proximity to the IMD. This is sometimes achieved by taping a simple magnet to the patient's chest. Such a practice operates with varying degrees of success. The downside risks associated with this practice includes the medical professional's lack of knowledge of the exact location, and status of the IMD, and the potential shifting or dislodging of the magnet during surgery as might be occasioned when the patient's body is manipulated during any medical procedure.

The reference to Lure et, al. (U.S. Pub. No. 2011/0160782) describes an attempt to resolve some of the same issues described, above, by disclosing a hand-held programming device that uses wireless RF signals to switch an IMD into, and out of, safety mode. Such a “hand-held” computer programming device is far more costly to manufacture than the invention as disclosed hereinafter, and cannot be easily stored or be made readily available in appropriate, and useful locations such as emergency rooms, operating rooms, first-responder vehicles, and nursing facilities. Furthermore, the invention as disclosed, hereinafter, allows for convenient removal and instant re-activation of an IMD to its normal and preferred settings thus obviating the need for re-interrogation or programming by either a hand-held or laptop-type device as has been the conventional practice heretofore.

Most importantly the prior art devices and practices typically cause an undesirable and permanent change in the programming of the IMDs, and which are effected by many of the devices taught in prior art, and typically those which use RF computer reprogramming. Those skilled in the art will readily recognize that permanently changing the internal programming of an IMD may leave a patient unprotected against potentially lethal heart arrhythmias or dangerous sub-optimal pacemaker performance. Further, pre-programming of an IMD typically does not allow for modifications of the programming window in the event that a surgery is postponed or prolonged. Of course, leaving a patient programmed to an alternate operational mode for any defined, unmonitored period of time prior to, or after surgery is dangerous given the inherent unpredictability of medical care.

A device and related methodology which avoids the detriments associates with the prior art devices and practices used heretofore is the subject matter of the present application.

### SUMMARY OF THE INVENTION

Therefore, a first aspect of the present invention is to provide a device for controlling an IMD, and which includes a housing defining an internal cavity, and which is releasably attached to an area of a patient's body, and which is near an IMD that is placed within the patient's body, and wherein the IMD, when energized, emits a first source of electromagnetic radiation, has an operational condition, and is of a predetermined type; a magnet mounted within the internal cavity, and which generates a second source of electromagnetic radiation, and which is effective in changing the operational condition of the IMD; an electronic sensor mounted within the internal cavity and which, when rendered operable, detects the first source of electromagnetic radiation emitted by the IMD, locates the IMD within the patient's body, identifies the type of the IMD within the patient's body, and further identifies the operational condition of the IMD; a signaling assembly borne by the housing, and further located within the internal cavity thereof, and which indicates the type of the IMD, the operational condition of the IMD, and the proper location for the magnet relative to the IMD so that the magnet can effect the operational condition of the IMD; an electrical circuit coupling the electronic sensor, and the signaling assembly, together; and a source of electrical power borne by the housing, and further located within the internal cavity thereof, and which is selectively electrically coupled to the electrical circuit so as to deliver electrical power to selectively energize the electronic sensor and signaling assembly

Another aspect of the present invention relates to a device for controlling an IMD and which includes a flexible housing which includes a main body having opposite top and bottom surfaces, and wherein the housing further defines an internal cavity, and wherein the bottom surface of the housing is at least partially adhesive, and wherein the bottom surface is operable to secure the housing on a given location of a patient's body, and which is in proximity to the IMD; a magnet mounted within the internal cavity of the housing, and which generates a source of electromagnetic radiation which is effective in operably controlling an operational condition of the IMD; an electronic sensor mounted within the internal cavity of the housing, and which is effective in detecting a source of electromagnetic radiation which is generated by the IMD so as to locate the IMD within a body cavity of a patient to be treated, and wherein the electronic sensor further determines from the emitted electromagnetic radiation generated by the IMD the operational condition of the IMD, and further identifies the type of the IMD; a first signaling assembly which is borne by the top surface of the housing and which further is located within the internal cavity thereof, and which is also operably coupled with the electronic sensor, and wherein the first signaling assembly provides a predetermined signal indicating the type of the IMD, and the operational condition of the IMD within the patient's body; a second signaling assembly which is borne by the top surface of the housing, and which is further located within the internal cavity thereof, and which is operably coupled with the electronic sensor, and wherein the second signaling assembly, when energized, emits a predetermined first audio signal which indicates the proper location for the magnet relative to the IMD so that the magnet may effect the operational condition of the IMD, and a second audio signal if the operational condition of the IMD changes; an electrical circuit mounted within the internal cavity of the housing, and which electrically and operably couples together the electronic sensor, and the first and second signaling assemblies; and a source of electrical power which is mounted within the internal cavity of the housing, and which further is electrically coupled to the electrical circuit.

Another aspect of the present invention is to provide a method for controlling an IMD, and which includes the steps of providing a flexible housing which includes a main body having opposite top and bottom surfaces, and wherein the housing further defines an internal cavity; depositing an adhesive coating on at least a portion of the bottom surface of the housing, and wherein the adhesive is operable to secure the housing on a given location on a patient's body and which is in proximity to an IMD which is located within the patient's body; positioning a magnet within the internal cavity of the housing, and which generates a source of electromagnetic radiation which is effective in operably controlling an operational condition of the IMD when the flexible housing is located in proximity to the IMD; positioning an electronic sensor within the internal cavity of the housing, and which is effective in detecting a source of electromagnet radiation which is generated by the IMD, and wherein the electronic sensor utilizes the electromagnetic radiation generated by the IMD to determine a location of the IMD within the body of the patient to be treated, the operational condition of the IMD, and the type of the IMD which is within the patient's body; providing a first signaling assembly which is borne by the top surface of the housing and which is further located within the internal cavity thereof, and which is operably coupled with the electronic sensor, and wherein the signaling assembly provides a predetermined signal which indicates the type and operational condition of the IMD within the body cavity of the patient to be treated; providing a second signaling assembly which is borne by the top surface of the housing and which is further located within the internal cavity thereof, and which is operably coupled with the electronic sensor, and which is further effective to generate a signal which indicates to a user a proper location for the magnet relative to the IMD, and which is located within the body of the patient to be treated; providing an electrical circuit, and orienting the electrical circuit within the internal cavity of the housing, and further coupling the electronic sensor, the first signaling assembly, and the second signaling assembly to the electrical circuit; selectively energizing the electronic sensor, the first signaling assembly, and the second signaling assembly by a source of electrical power which is borne by the housing, and further located within the internal cavity thereof, and which is further electrically coupled to the electrical circuit; delivering the source of electrical power to energize the electrical circuit which is coupled to the electronic sensor, the first signaling assembly, and the second signaling assembly; emitting a continuous signal with the energized second signaling assembly to indicate the delivery of the source of electrical power to the electrical circuit; positioning the housing over the body of the patient to be treated so as to locate the position of the IMD within the patient's body, and to further properly position the magnet relative to the IMD so as to effect an operational condition of the IMD; determining the location of the IMD by the electronic sensor; changing the operational status of the IMD by way of the electromagnetic radiation which is generated by the magnet; determining the operational status of the IMD, and the type of the IMD by the electronic sensor; adhering the housing in proximity to the IMD by way of the adhesive coating which is deposited on the bottom surface of the housing so as to effect a continual change in the operational condition of the IMD; and determining any change in the operational status of the IMD with the electronic sensor and which has been effected by the electromagnetic radiation which is emitted by the magnet.

### BRIEF DESCRIPTION OF THE DRAWINGS

Preferred embodiments of the invention are described below with reference to the following accompanying drawings.

FIG. 1 is a fanciful, fragmentary view of a patient's body and showing the patient's heart, and an implanted medical device (IMD) in phantom lines.

FIG. 2 is a schematic representation of the electrical circuit, and operable relationship of the components of the present invention.

FIG. 3 is a greatly simplified, top, plan view of the present invention, and which shows several underlying structures in phantom lines.

FIG. 4 is a transverse, vertical sectional view of the present invention and which is taken from a position along line 4-4 of FIG. 3.

FIG. 5 is a transverse, vertical sectional view of the present invention and which is taken from a position along line 5-5 of FIG. 3.

FIG. 6 is a fanciful, fragmentary view of a patient's body having an implanted medical device (IMD) during one step of methodology of present invention.

FIG. 7 is a fanciful, fragmentary view of a patient's body during another step of the disclosed methodology.

### DETAILED DESCRIPTION OF THE PREFERRED EMBODIMENTS

This disclosure of the invention is submitted in furtherance of the constitutional purposes of the US Patent laws “to promote the progress of science and useful arts” (Article 1, Section 8).

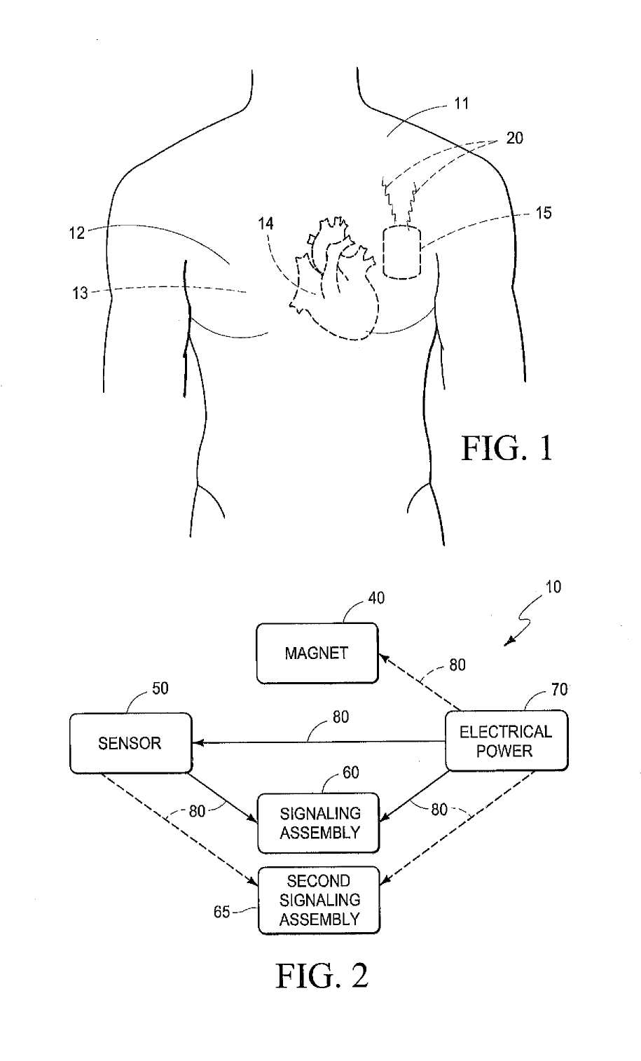

The present invention 10 is generally indicated by the numeral 10 in FIG. 2 and following. The present invention, which concerns a device for controlling an Implanted Medical Device [IMD] is useful for treating a human patient, which is generally indicated by the numeral 11 in FIG. 1. As seen in FIG. 1, the human patient 11 has a chest and torso region 12. The chest or torso region also defines an internal chest cavity 13. The patient's heart 14, which is shown in phantom lines in FIG. 1, is located within the internal chest cavity. Still further, an implanted medical device, which is only generally indicated by the numeral 15, is located within the internal chest cavity and which is further operable to emit electromagnetic radiation 20, as will be described below, and which controls a function of the heart 14 of the patient 11.

It should be understood from reading the background of the invention that the implanted medical device (IMD) 15 is selected from the group which includes pacemakers and defibrillators. Still further, it should be understood that the selected IMD has an operational condition which may constitute one of several operational states. These several states would include a first state or condition and where the IMD is operational, and affecting the patient's body 11; an inoperable condition; and a standby condition. As will be appreciated from the study of the drawings, and as is well known in the art, the implanted medical device 15, when rendered operational, generates electromagnetic radiation 20 which can be detected from a location that is external to the patient's body 11. Those skilled in the art will also recognize that an implanted medical device 15 is sometimes difficult to locate within the chest cavity 13 of the torso 12, and the present invention 10 provides a convenient methodology whereby a treating physician may conveniently locate the implanted medical device 15, and then place it into a desired operational state or condition, as will be discussed in greater detail, hereinafter.

The present invention 10 includes a flexible housing, which is generally indicated by the numeral 30. The flexible housing has a main body 31, having a top surface 32, and an opposite bottom surface 33. The top and bottom surfaces are spaced a predetermined distance apart, and define an internal cavity 34 which is located therebetween. As seen in the vertical sectional views of FIGS. 4 and 5, an adhesive coating 35 is applied to the bottom surface 33. The adhesive coating is protected until use by a disposable release sheet 36, of traditional design, and which is disposed in covering relation over the adhesive coating 35. As should be understood, the bottom surface 33 is operable to releasably, adhesively secure the housing 30 on a given location of the patient's body 11, and which is in proximity to the IMD 15.

The internal cavity 34 of the housing 30 encloses a magnet 40; an electronic sensor 50; a first signaling assembly 60; a second signaling assembly 65; a source of electrical power 70; and an electrical circuit 80 which electrically and operably couples the aforementioned components together. The internal cavity 34 of the housing 30 may be at least partially accessible from the top surface 32, of the housing 30 by means of an accessibility port 37 of a type which is well known in the art.

The magnet 40 which is mounted within the internal cavity 34 generates a second source of electromagnetic radiation 41, and which is effective in changing the operational condition of an IMD 15 when the magnet 40 is placed in proximity to the IMD 15. In one form of the invention, the magnet 40 may continually emit a source of electromagnetic radiation 41, or in another possible form of the invention may selectively emit a source of electromagnetic radiation 41. When a selectively energizable magnet is used to emit electromagnetic radiation 41, the magnet 40 will be selectively electrically coupled to the electrical circuit 80 and which is described in further detail, hereinafter, so as to become energized. Furthermore, the magnet 40, as used in the present invention may be fabricated in a manner so as to be flexible, and render it useful in conforming to the contour of the patient's body 11. The magnet is typically fabricated in the shape of a ring. However other shapes may be used.

Also mounted within the internal cavity 34 of the housing 30 of the present invention 10 is an electronic sensor 50 which is effective in detecting the source of electromagnetic radiation 20 which is generated by the IMD 15. The electronic sensor 50 is capable of determining from the electromagnetic radiation 20 which is emitted by the IMD 15, the operational condition of the IMD 15, and the type of the IMD 15 which is implanted within the patient.

The present invention 10 further includes a first signaling assembly 60 which is borne, at least in part, by the top surface 32 of the housing 30, and is further located, in part, within the internal cavity 34. The first signaling assembly 60 is operably electrically coupled with the electronic sensor 50, and wherein the first signaling assembly 60 provides a predetermined signal 61 indicating the type of the IMD 15 and the operational condition of the IMD 15 which was determined by the electronic sensor 50 when the invention is brought near to the IMD 15.

Although not essential, the present invention 10 may also include a second signaling assembly 65 which is borne, at least in part, by the top surface 32 of the housing 30, and is further located, in part, within the internal cavity 34. The second signaling assembly 65 is electrically operably coupled with the electronic sensor 50, and wherein the second signal assembly 65, when energized emits a predetermined first signal 66, to indicate to a user the appropriate location for the magnet 40, relative to the IMD 15, so that the magnet 40 may affect the operational condition of the IMD 15. The second signaling assembly 65 further is rendered operable when energized to emit a second signal 66 when the operational condition of the IMD 15 changes. The second signaling assembly 65 may further provide yet another signal 66 when the source of electrical power 70 is initially electrically coupled to the electrical circuit 80. The signals 61 and 66 which are generated by the first signaling assembly 60, and the second signaling assembly 65, can be visual, audio and/or a combination of both.

An electrical circuit 80, which is only generally depicted in FIG. 1, is also mounted within the internal cavity 34 of the housing 30, and is electrically and operably coupled to the electronic sensor 50, the first signaling assembly 60, the second signaling assembly 65, and the source of electrical power 70. The source of electrical power 70 is borne by the housing 30, and provides a source of electrical power to energize the sensor 50; first signaling assembly 60; second signaling assembly 65; and on occasion an electro-magnet 40, through the electrical circuit 80. The source of electrical power 70 typically comprises a battery which is mounted within the internal cavity 34. The battery may be of a single-use type, or in some forms of the invention may be rechargeable.

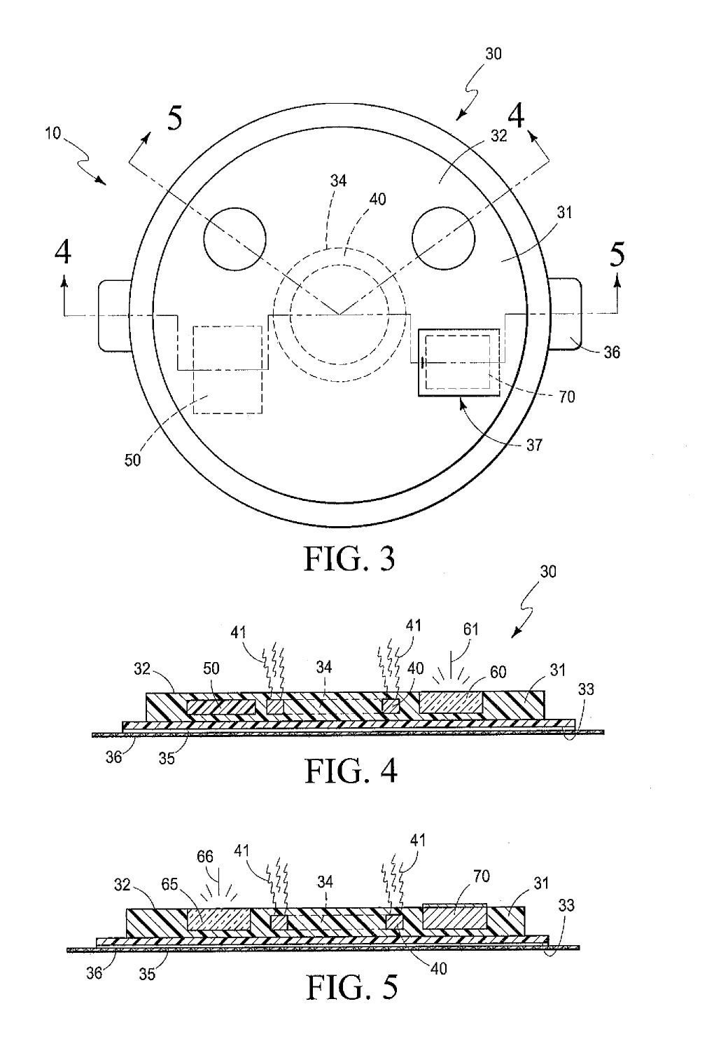

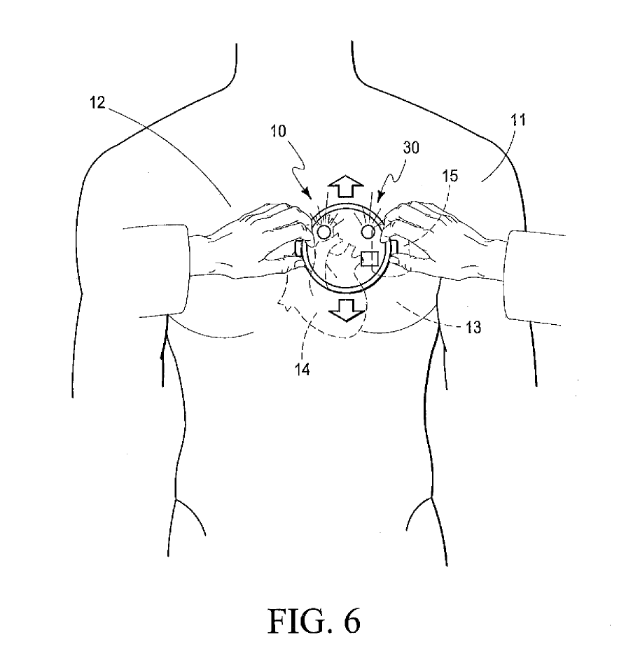

Furthermore, the present invention 10 includes a method for controlling an IMD 15 and which includes a first step of providing a flexible housing 30 which includes a main body 31, having a top surface 32, and bottom surface 33. The respective surfaces define an internal cavity 34 therebetween (FIGS. 3 and 4). The method includes another step of depositing an adhesive coating 35 on at least a portion of the bottom surface 33 of the housing 30, and wherein the adhesive 35 is operable to secure the housing 30 on a given location on a patient's body 11, and which is in proximity to an IMD 15 that is located within the internal chest cavity 13 of the patient's body 11 (FIGS. 4 and 5).

The method involves yet another step of positioning a magnet 40 within the internal cavity 34 of the housing 30 and which generates a source of electromagnetic radiation 41 (FIG. 5) which is effective in operably controlling the operational condition of the IMD 15, when the flexible housing 30, is located in proximity to the IMD 15.

The method further includes another step of positioning an electronic sensor 50 within the internal cavity 34 of the housing 30 and which is effective in detecting the source of electromagnetic radiation 20 which is generated by the IMD 15. The electronic sensor 50 utilizes the electromagnetic radiation 20 generated by the IMD 15 to determine the location of the IMD 15 within the body of the patient 11, the operational condition of the IMD 15, and the type of the IMD 15 in the patient's body 11.

The method further includes yet another step of providing a first signaling assembly 60 which is borne by the housing 30, and which is further located within the internal cavity 34 of the housing 30. The first signaling assembly 60 is operably coupled with the electronic sensor 50, and further provides a predetermined signal 61 which indicates the type and operational condition of the IMD 15 within the body cavity 13 of the patient 11 to a physician employing the invention 10.

The method of the present invention further includes a step of providing a second signaling assembly 65 which is borne by the housing 30, and which is further located within the internal cavity 34 of the housing 30. The second signaling assembly 65 is operably coupled to the electronic sensor 50, and is further effective in generating a signal 66 which indicates to a user, such as an attending physician, the proper location for the magnet 40 relative to the IMD 15, so as to effect a change in its operational condition, and which is located within the chest cavity 13 of a patient 11 to be treated.

The method further includes still another step of providing an electrical circuit 80 (FIG. 2), and orienting the electrical circuit 80 within the internal cavity 34 of the housing 30. The electrical circuit 80 is coupled to each of the electronic sensor 50, the first signaling assembly 60, and the second signaling assembly 65. The method of the present invention includes still another step of selectively energizing the electronic sensor 50, the first signaling assembly 60, and the second signaling assembly 65 by the source of the electrical power 70 which is borne by the housing 30, and which is further located within the internal cavity 34. The source of electrical power 70 is selectively electrically coupled to the electrical circuit 80. The method includes yet another step of delivering the source of the electrical power 70 to energize the electrical circuit 80 and which is further coupled to the electronic sensor 50, the first signaling assembly 60, and the second signaling assembly 65. Once energized, the second signaling assembly 65 emits a predetermined signal 66 to indicate the delivery of the source of electrical power 70 to the electrical circuit 80, to a user employing the invention 10.

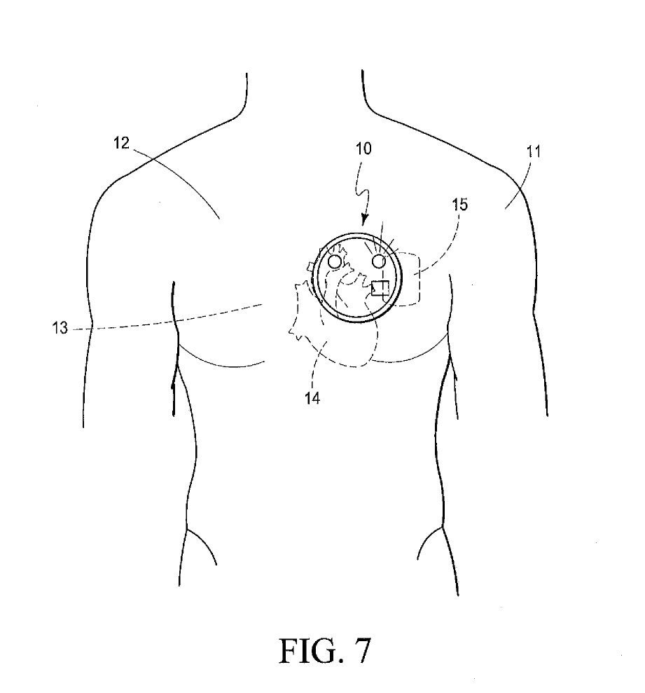

The method includes still another step of positioning the housing 30 over the body of a human patient 11 to be treated so as to locate the position of the IMD 15 within the internal chest cavity 13 of the patient 11, and to further properly position the magnet 40 relative to the IMD 15 so as to effect the operational condition of the IMD 15 (FIG. 7). This step involves determining the location of the IMD 15 by the electronic sensor 50. When the invention is appropriately oriented, the operational condition of the IMD 15 is changed by way of the electromagnetic radiation 41 which is emitted by the magnet 40 when it is placed within proximity to the IMD 15. The method further includes still another step of determining the operational status of the IMD 15, and the type of the IMD 15 by the use of the electronic sensor 50. When appropriately operated, the method includes another step of adhering the housing 30 in proximity to the IMD 15, and which is located within the internal chest cavity 13 of the human patient 11 by way of the adhesive coating 35 which is deposited on the bottom surface 33 of the housing 30. This fixed orientation of the magnet 40 effects a continual change in the operational condition of the IMD 15. The method then includes another step of determining any change in the operational status of the IMD 15 with the electronic sensor 50 in the event that the magnet is displaced from the patient's body 11.

The method of the present invention may include another step of silencing, or stopping the signal 66 which is emitted by the second signaling assembly 65 when the IMD 15 is located, and the magnet 40 is properly positioned in proximity to the IMD 15 so as to change the operational condition of the IMD. Additionally, still another step of the method may include emitting a signal 61 with the first signaling assembly 60 to indicate the operational status of the IMD 15, and to further indicate the type of the IMD 15 located within the patient's body 11. The method may further include a step of emitting a signal 66 from the second signaling assembly 65, to indicate a change in the operational condition of the IMD 15. The method may also further include a step of de-energizing the signal 61 from the first signaling assembly 60 so as to indicate a change in the operational status of the IMD 15.

### Operation

The operation of the described embodiment of the present invention is believed to be readily apparent and is briefly summarized at this point.

In its broadest aspect, a device 10 for controlling an implanted medical device 15 includes a housing 30, defining an internal cavity 34, and which is releasably attached to an area of a patient's body 11, and wherein the IMD 15, when energized, emits a first source of electromagnetic radiation 20, and further has an operational condition, and is of a predetermined type of IMD. The present invention 10 also includes a magnet 40 which is mounted within the internal cavity 34, and which further generates a second source of electromagnetic radiation 41, and which is effective in changing the operational condition of the IMD 15. The invention further includes an electronic sensor 50 mounted within the internal cavity 34, and which when rendered operable detects the first source of electromagnetic radiation 20, which is emitted by the IMD 15, locates the IMD 15 within the patient's body 11, identifies the type of IMD 15 within the patient's body, and further identifies the operational condition of the IMD 15. The invention also includes a signaling assembly 60, which is borne by the housing 30, and is further located within the internal cavity 34 thereof. The signaling assembly when rendered operable indicates the type of the IMD 15, the operational condition of the IMD 15, and the proper location for the magnet 40 relative to the IMD 15, so that the magnet 40 can affect an operational condition of the IMD 15. The present invention also includes an electrical circuit 80, which couples the electronic sensor 50, and signaling assembly 60, together. Further, the invention includes a source of electric power 70 which is mounted within the internal cavity 34, and which further is selectively electrically coupled to the electrical circuit 80 so as to selectively energize the electronic sensor 50, and signaling assembly 60, so as to provide effective signals for a user of the invention who is employing it to effect a change in the IMD 15.

Therefore, it will be seen that the present invention provides a convenient means whereby an attending physician may effect a change in the operational condition of an IMD 15, in a manner not possible heretofore. The present invention is easy to use, can effect a change in the operational condition of an IMO in a manner so as to allow a surgical intervention to proceed with minimal risks, and further provides minimum health-related problems for a patient who is in need of an IMD because of a pre-existing coronary or other medical condition warranting the IMD.

In compliance with the statute, the invention has been described in language more or less specific as to structural and methodical features. It is to be understood, however, that the invention is not limited to the specific features shown and described since the means herein disclosed comprise the preferred forms of putting the invention into effect. The invention is, therefore, claimed in any of its forms or modifications within the proper scope of the appended claims appropriately interpreted in accordance with the doctrine of equivalence.

We claim:

1. A device for controlling an implanted medical device (IMD) comprising:
a housing defining an internal cavity, and which is releasably attached to an area of a patient's body and which is near an IMD that is placed within the patient's body, and wherein the IMD, when energized, emits a first source of electromagnetic radiation, has an operational condition, and is of a predetermined type;
a magnet mounted within the internal cavity, and which generates a second source of electromagnetic radiation and which is effective in changing the operational condition of the IMD;
an electronic sensor mounted within the internal cavity and which, when rendered operable, detects the first source of electromagnetic radiation emitted by the IMD, locates the IMD within the patient's body, identifies the type of the IMD within the patient's body, and further identifies the operational condition of the IMD;
a signaling assembly borne by the housing and further located within the internal cavity thereof, and which indicates the type of the IMD, the operational condition of the IMD, and the proper location for the magnet relative to the IMD so that the magnet can effect the operational condition of the IMD;
an electrical circuit coupling the electronic sensor and signaling assembly together; and
a source of electrical power mounted within the internal cavity, and which is selectively electrically coupled to the electrical circuit so as to deliver electrical power to selectively energize the electronic sensor and signaling assembly.

2. A device as claimed in claim 1, and wherein the housing has top and bottom surface, and wherein an adhesive coating is applied to at least a portion of the bottom surface of the housing, and wherein the adhesive coating releasably secures the housing in a predetermined location on the patient's body and in proximity to the IMD so that the second source of electromagnetic radiation which is emitted by the magnet is effective in controlling the operational condition of the IMD.

3. A device as claimed in claim 1, and wherein the magnet continually emits the second source of electromagnetic radiation.

4. A device as claimed in claim 1, and wherein the magnet selectively emits the second source of electromagnetic radiation.

5. A device as claimed in claim 1, and wherein the operational condition of the IMD is selected from the group comprising an operational condition; an inoperable condition; and a stand-by condition.

6. A device as claimed in claim 1, and wherein the type of the IMD is selected from the group which include pace-makers and defibrillators.

7. A device as claimed in claim 1, and wherein the signaling assembly includes a first and second signaling assembly, and wherein the first signaling assembly, when energized, indicates the type and operational condition of the IMD; and
a second signaling assembly which, when energized, indicates a proper location for the magnet relative to the IMD so that the second source of electromagnetic radiation is effective in changing the operational condition of the IMD, and if the operational condition of the IMD changes.

8. A device as claimed in claim 7, and wherein the first and second signaling assemblies individually generate signals which can be visual, audio and/or both.

9. A device as claimed in claim 1, and wherein the source of electrical power comprises a battery which is borne by the housing and is further located within the internal cavity thereof.

10. A device as claimed in claim 1, and wherein the housing is flexible.

11. A device for controlling an IMD comprising:
a flexible housing which includes a main body having opposite top and bottom surfaces, and wherein the housing further defines an internal cavity, and wherein the bottom surface is at least partially adhesive, and wherein the bottom surface is operable to secure the housing on a given location of a patient's body, and which is in proximity to the IMD;
a magnet mounted within the internal cavity of the housing, and which generates a source of electromagnetic radiation which is effective in operably controlling an operational condition of the IMD;
an electronic sensor mounted within the internal cavity of the housing, and which is effective in detecting a source of electromagnetic radiation which is generated by the IMD so as to locate the IMD within a body cavity of a patient to be treated, and wherein the electronic sensor further determines from the emitted electromagnetic radiation generated by the IMD the operational condition of the IMD, and further identifies the type of the IMD;
a first signaling assembly which is borne by the top surface of the housing and further located within the internal cavity thereof, and which is operably coupled with the electronic sensor, and wherein the first signaling assembly provides a predetermined signal indicating the type of the IMD, and the operational condition of the IMD within the body of the patient;
a second signaling assembly which is borne by the top surface of the housing and further located within the internal cavity thereof, and which is operably coupled with the electronic sensor, and wherein the second signaling assembly, when energized, emits a predetermined first audio signal which indicates the proper location for the magnet relative to the IMD so that the magnet may effect the operational condition of the IMD, and a second audio signal if the operational condition of the IMD changes;
an electrical circuit mounted within the internal cavity of the housing, and which electrically and operably couples together the electronic sensor, and the first and second signaling assemblies; and
a source of electrical power which is mounted within the internal cavity, and which further is electrically coupled to the electrical circuit.

12. A device as claimed in claim 11, and wherein the magnet is flexible.

13. A device as claimed in claim 12, and wherein the magnet is a ring magnet.

14. A device as claimed in claim 12, and wherein the magnet further comprises a selectively energizable electromagnet which is coupled to the electrical circuit.

15. A device as claimed in claim 11, and wherein the flexible housing is fabricated from a reusable material.

16. A device as claimed in claim 11, and wherein the bottom surface of the housing is at least partially covered by an adhesive coating.

17. A device as claimed in claim 11, and wherein the internal cavity is at least partially accessible.

18. A device according to claim 11, and wherein the second signaling assembly is further operable to provide an audio signal when the source of electrical power is initially electrically coupled to the electrical circuit.

19. A method for controlling an IMD, comprising:
providing a flexible housing which includes a main body having opposite top and bottom surfaces, and wherein the housing further defines an internal cavity;
depositing an adhesive coating on at least a portion of the bottom surface of the housing, and wherein the adhesive is operable to secure the housing on a given location on a patient's body and which is in proximity to an IMD which is located within the patient's body;
positioning a magnet within the internal cavity of the housing, and which generates a source of electromagnetic radiation which is effective in operably controlling an operational condition of the IMD when the flexible housing is located in proximity to the IMD;
positioning an electronic sensor within the internal cavity of the housing, and which is effective in detecting a source of electromagnet radiation which is generated by the IMD, and wherein the electronic sensor utilizes the electromagnetic radiation generated by the IMD to determine a location of the IMD within the body of the patient to be treated, the operational condition of the IMD, and the type of the IMD which is within the patient's body;
providing a first signaling assembly which is borne by the top surface of the housing and further located within the internal cavity thereof, and which is operably coupled with the electronic sensor, and wherein the signaling assembly provides a predetermined signal which indicates the type and operational condition of the IMD within the body cavity of the patient to be treated;
providing a second signaling assembly which is borne by the top surface of the housing and further located within the internal cavity thereof, and which is operably coupled with the electronic sensor, and which is further effective to generate a signal which indicates to a user a proper location for the magnet relative to the IMD and which is located within the body of the patient to be treated;
providing an electrical circuit and orienting the electrical circuit within the internal cavity of the housing, and further coupling the electronic sensor, the first signaling assembly, and the second signaling assembly to the electrical circuit;
selectively energizing the electronic sensor, the first signaling assembly, and the second signaling assembly by a source of electrical power which is borne by the housing and further located within the internal cavity thereof, and which is further electrically coupled to the electrical circuit;
delivering the source of electrical power to energize the electrical circuit which is coupled to the electronic sensor, the first signaling assembly, and the second signaling assembly;
emitting a continuous signal with the energized second signaling assembly to indicate the delivery of the source of electrical power to the electrical circuit;
positioning the housing over the body of the patient to be treated so as to locate the position of the IMD within the patient's body, and to further properly position the magnet relative to the IMD so as to effect an operational condition of the IMD;
determining the location of the IMD by the electronic sensor;
changing the operational status of the IMD by way of the electromagnetic radiation which is generated by the magnet;
determining the operational status of the IMD, and the type of the IMD by the electronic sensor;
adhering the housing in proximity to the IMD by way of the adhesive coating which is deposited on the bottom surface of the housing so as to effect a continual change in the operational condition of the IMD; and
determining any change in the operational status of the IMD with the electronic sensor and which has been effected by the electromagnetic radiation which is emitted by the magnet.

20. A method as claimed in claim 19, and further comprising silencing the signal emitted by the second signaling assembly when the IMD is located, and the magnet is properly positioned in proximity to the IMD.

21. A method as claimed in claim 19, and further comprising emitting a signal with the first signaling assembly to indicate the operational status of the IMD, and to further indicate the type of the IMD which is within the patient's body.

22. A method as claimed in claim 19, and further comprising emitting a signal with the second signaling assembly to indicate a change in the operational condition of the IMD.

23. A method as claimed in claim 19, and further comprising deenergizing the signal generated by the first signaling assembly so as to indicate a change in the operational status of the IMD.
